(19) United States
(12) Patent Application Publication
(10) Pub. No.: US 2015/0144792 A1
(43) Pub. Date: May 28, 2015

(54) Vibrational Spectroscopic Techniques for Classifying Chronic Pain States

(71) Applicant: Ethos Research & Development, Fairfield, OH (US)

(72) Inventor: Joshua A. Gunn, Fairfield, OH (US)

(21) Appl. No.: 14/553,135

(22) Filed: Nov. 25, 2014

Related U.S. Application Data
(60) Provisional application No. 61/908,388, filed on Nov. 25, 2013.

(51) Int. Cl. A61B 5/00; G01N 21/35; G01N 21/65

(52) U.S. Cl. CPC A61B 5/4824; A61B 5/0075; G01N 21/65; G01N 21/35; G01N 2021/3595; G01N 2201/125
USPC 250/339.08; 356/301

(57) ABSTRACT

Methods and systems for diagnosing a chronic pain syndrome in a subject are provided. The methods and systems include approaches for identifying a diagnosis based on the vibrational spectra of biological samples.

### CROSS-REFERENCE TO RELATED APPLICATIONS

This application claims the benefit under 35 U.S.C. §119(e) to U.S. Provisional Application 61/908,388, filed Nov. 25, 2013 and entitled “Vibrational Spectroscopic Techniques for Classifying Chronic Pain States,” which is hereby incorporated by reference in its entirety.

### TECHNICAL FIELD OF THE INVENTION

Exemplary embodiments relate to methods and systems for classifying chronic pain disorders using vibrational spectra from approaches such as Fourier transform infrared spectroscopy and Raman spectroscopy.

### BACKGROUND OF THE INVENTION

Traditional approaches to the differential diagnosis of chronic pain syndromes are limited in accuracy and reliability due to a lack of knowledge about the pathophysiology of the various diseases that produce chronic pain. Due to its often unclear etiology, complex natural history, and poor response to traditional therapies, chronic pain is also extremely difficult to manage. The early and accurate differential diagnosis of chronic pain can result in better patient outcomes and reduced cost to the healthcare system.

### BRIEF SUMMARY OF THE INVENTION

Accordingly, it is a primary object of various embodiments of the invention to provide methods and systems that can improve diagnoses of chronic pain syndromes, as well as to provide additional information to health care practitioners for improving the care of individuals who experience various types of chronic pain.

As discussed herein, the presently disclosed systems and methods relate to the identification of certain chronic pain disorders by way of performing a vibrational spectrum analysis on a biological sample from a test subject.

In some embodiments, the invention provides methods for diagnosing a chronic pain syndrome in a test subject, the method comprising: obtaining a vibrational spectrum for a biological sample from the test subject; and classifying the spectrum as representative of one or more candidate chronic pain syndromes.

In some embodiments, the invention provides a system for diagnosing a chronic pain syndrome in a test subject, the system comprising: one or more processors configured to execute one or more modules; and a memory storing the one or more modules, the modules comprising instructions for: obtaining a vibrational spectrum of a biological sample; calculating one or more scores assessing whether the test subject should be diagnosed with one or more candidate chronic pain syndromes from the output of a model, wherein the model input comprises the vibrational spectrum; and providing the one or more scores. In some embodiments, the system is linked to a device configured to generate a vibrational spectrum based on a biological sample, e.g., a spectrometer configured to obtain a vibrational spectrum of a biological sample. The spectrometer may be an infrared or Raman spectrometer.

Additional objects and advantages of the invention will be set forth in part in the description that follows. It is to be understood that both the foregoing general description and the following detailed description are exemplary and explanatory only and are not restrictive of the invention, as claimed.

### BRIEF DESCRIPTION OF THE DRAWINGS

Reference will now be made to the accompanying drawings showing example embodiments of this disclosure. In the drawings:

FIG. 1 is a block diagram of an exemplary system for classifying chronic pain states, consistent with embodiments of the present disclosure.

FIG. 2 is a flowchart representing exemplary methods for developing a classifier model, consistent with embodiments of the present disclosure.

FIG. 3 is a flowchart representing exemplary methods for classifying a test subject using a vibrational spectrum, consistent with embodiments of the present disclosure.

### DETAILED DESCRIPTION

The term “classification” or “classify” includes, for example the association of an individual, sample, and/or spectrum with a particular disease, biological condition or chronic pain state.

The term “diagnose” includes, for example, a conclusion that an individual has a particular disease or biological condition, or lack thereof, which is in many cases based on a classification of a sample and/or spectrum.

The term “user” can refer to, for example, (i) the individual operating the computing systems of certain embodiments to obtain a classification and/or (ii) the individual receiving the classification of the biological sample. Individual (i) and individual (ii) may be the same or different individuals. The user can be, for example, a computer or a person.

The term “chronic pain syndrome” can refer to any of a number of diagnoses that can be associated with the umbrella term chronic pain syndrome. The term “candidate chronic pain syndrome” can refer to a possible diagnosis. Examples of chronic pain syndromes are given in the following paragraph. The “chronic pain state” of a particular sample can refer to the status of a particular candidate chronic pain syndrome for a sample. A chronic pain state can, for example, refer to a stage in the progress of the chronic pain disorder, such as, for example, a lack of the disorder, a nascent instance of the disorder, a mature instance of the disorder, or an abated instance of the disorder. A chronic pain state can, for example, refer to the magnitude of the disorder, such as, for example, mild, moderate, or severe. A chronic pain state can, for example, refer to the state of being diseased or healthy.

In certain embodiments, the candidate chronic pain syndromes comprise or consist of fibromyalgia, myofascial pain syndrome, complex regional pain syndrome, osteoarthritis, rheumatoid arthritis, and chronic fatigue syndrome, and Post-Traumatic Stress Disorder (PTSD). In certain embodiments, the candidate chronic pain syndromes include one or more musculoskeletal and/or neuropsychological diseases, such as, for example, PTSD, hernias (e.g., obturator, sciatic, inguinal, femoral, perineal, spigelian, umbilical), neoplasia of the spinal cord or sacral nerves, mononeuropathy and nerve entrapment, abdominal epilepsy, abdominal migraines, pelvic floor pain syndrome, rectus abdominis pain, faulty posture and chronic pelvic pain, bipolar disorders and depression, chronic visceral pain syndrome, chronic fatigue syndrome, or substance abuse. In certain embodiments, the candidate chronic pain syndromes include one or more reproductive system disorders, such as, for example, adenomyosis, adhesions, adnexal cysts, cervical stenosis, dyspareunia, endocervical and endometrial polyps, endometriosis and endosalpingiosis, uterine leiomyomas, ovarian retention syndrome, ovarian remnant syndrome, pelvic varicosities and pelvic congestion syndrome, vulvodynia, pelvic floor relaxation disorders, or accessory and supernumerary ovaries. In certain embodiments, the candidate chronic pain syndromes include one or more urinary system disorders, such as, for example, chronic and/or recurrent urinary tract infections, urolithiasis, pelvic floor dysfunction, urethral diverticulum, or chronic urethral syndrome. In certain embodiments, the candidate chronic pain syndromes include one or more gastrointestinal system disorders, such as, for example, chronic intermittent bowel obstruction, colitis, chronic constipation, diverticular disease, inflammatory bowel disease, Irritable bowel syndrome, or peritoneal cysts. In certain embodiments, the candidate chronic pain syndromes include one or more other conditions, such as, for example, lateral epicondylitis, lumbar degenerative disk disease, lumbar facet arthropathy, lumbar spondylolysis and spondylolisthesis, mechanical low back pain, medial epicondylitis, meralgia paresthetica, mononeuritis multiplex, morton neuroma, myofascial pain, neoplastic brachial plexopathy, neoplastic lumbosacral plexopathy, osteoarthritis, osteoporosis and spinal cord injury, piriformis syndrome, plantar fasciitis, radiation-induced brachial plexopathy, radiation-induced lumbosacral plexopathy, rotator cuff disease, spasticity, thoracic outlet syndrome, traumatic brachial plexopathy, trochanteric bursitis, achilles tendoninjuries and tendonitis, adhesive capsulitis, brachial neuritis, carpal tunnel syndrome, cervical disc disease, cervical myofascial pain, cervical spondylosis, cervical sprain and strain, complex regional pain syndrome, rheumatoid arthritis, and fibromyalgia.

Vibrational spectroscopic techniques, such as Raman spectroscopy or Fourier transform infrared spectroscopy (FT-IR), provide analytical tools giving information about the molecular composition and structure of a given sample. Biological samples (including, for example, whole blood, serum, plasma, urine, saliva, and buccal swabs) can produce spectra that provide information about the composition and structure of constituent biomolecules such as, for example, nucleic acids, proteins, lipids (e.g., fatty acids, phospholipids, sterols, and triglycerides), and carbohydrates. The pathological changes associated with disease progression can be detectable in the spectra of such samples. Vibrational spectra obtained from diseased patients can exhibit small but reproducible differences when compared to samples from healthy patients. Thus, vibrational spectroscopy can provide a non-invasive, non-destructive, and/or inexpensive diagnostic tool.

The differences that can be detectable in the spectra of diseased samples when compared to healthy samples are often not visible to the human eye. Accordingly, chemometric analysis using pattern recognition techniques can be applied to process the spectral data.

By first analyzing a “training set” of known samples, a classifier can identify unique characteristics of certain disease states, which can then be used to identify test samples. Each diagnosis of interest can first be validated by analyzing a certain number of samples exhibiting the disease or diagnosis of interest so that the chemometric platform has sufficient data to identify unique spectral characteristics of the given disease.

Reference will now be made in detail to the exemplary embodiments implemented according to the present disclosure, certain examples of which are illustrated in the accompanying drawings. Wherever possible, the same reference numbers will be used throughout the drawings to refer to the same or like parts.

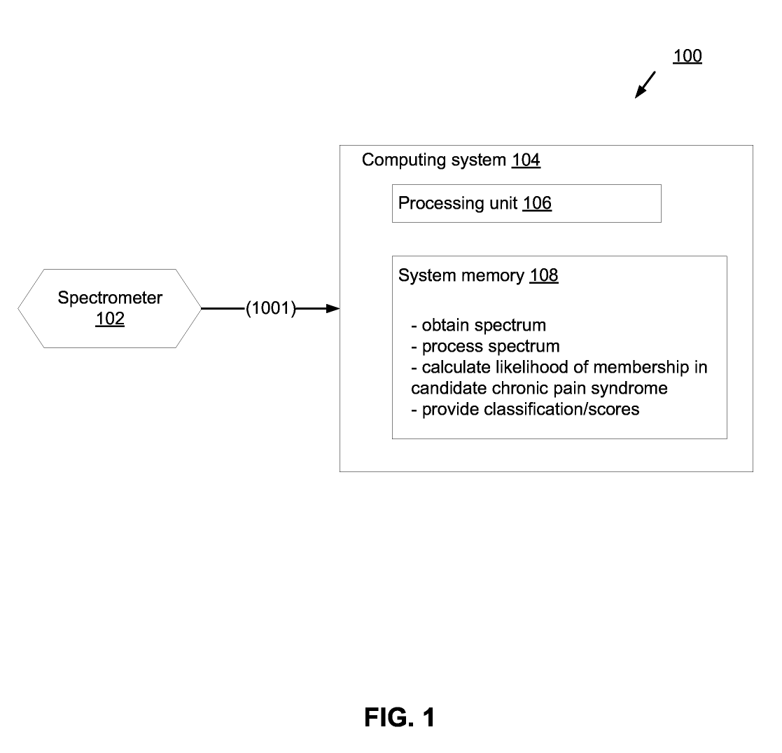

FIG. 1 is a block diagram of an exemplary system for classifying chronic pain states 100. Exemplary system 100 classifies chronic pain states based on a vibrational spectrum generated by spectrometer 102.

Spectrometer 102 can be, for example, configured for Raman spectroscopy or FT-IR. Spectrometer 102 can be configured to obtain data using, for example, attenuated total reflection (ATR), transmission, or reflectance. In certain embodiments, spectrometer 102 will be configured to analyze a series of samples in an autosampler, such as an autosampler comprising a 96-well plate.

Spectral data generated by spectrometer 102 can be provided to computing system 104 by a route 1001 such as, for example, via a network connection, a removable disk, or a local data connection. Computing system 104 includes a processing unit 106 comprising one or more processors, and a system memory 108 that stores one or more modules. The one or more modules can comprise, for example, modules for obtaining a vibrational spectrum, processing a vibrational spectrum, classifying a spectrum based on reference spectra, calculating a score assessing membership in a candidate chronic pain syndrome, providing a classification and/or one or more scores assessing membership in a chronic pain syndrome.

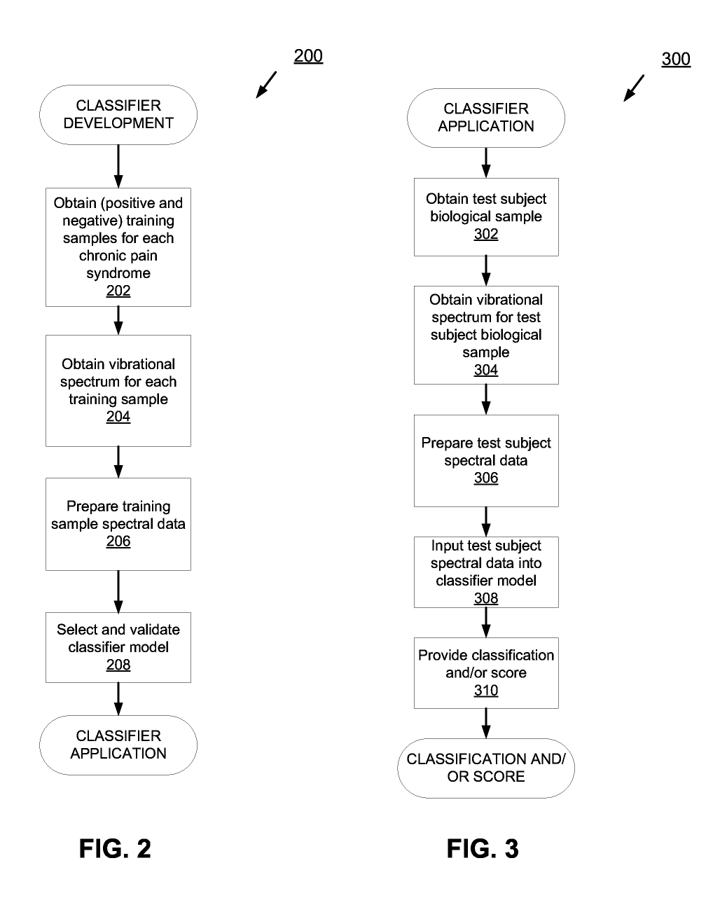

FIG. 2 is a flowchart representing exemplary methods for developing a classifier model, consistent with embodiments of the present disclosure. Referring to FIG. 2, it will be readily appreciated by one of ordinary skill in the art that the illustrated procedure can be altered to delete steps and/or include additional steps. The procedure begins by obtaining positive and negative training samples (for example, healthy samples and diseased samples for each candidate chronic pain syndrome) 202. The training samples can be biological samples drawn from various classes and processed using various techniques as described below for step 302.

The term “diseased” can, for example, refer to the state of a sample drawn from an individual who has been positively diagnosed with, for example, a chronic pain syndrome. The term “healthy” can, for example, be used to refer to the state of a sample drawn from an individual that has been diagnosed as not having, for example, a chronic pain syndrome, or an individual who has not been diagnosed with a chronic pain syndrome.

Next, a vibrational spectrum is obtained for each training sample 204. The vibrational spectrum can be obtained using any of the approaches as described below for step (304).

Next, the training sample vibrational spectra can be prepared 206. The spectra can be prepared using any of the approaches as described below for step 306.

Next, a classifier model can be selected and validated based on its performance with respect to the training samples 208. The model can be selected and validated based on the processing and model implementation that best discriminates between healthy and diseased training samples. The model may learn or retain some representation of a spectral signature for each candidate chronic pain syndrome.

FIG. 3 is a flowchart representing exemplary methods for classifying a test subject using a vibrational spectrum, consistent with embodiments of the present disclosure. As with FIG. 2, it will be readily appreciated by one of ordinary skill in the art that the illustrated procedure can be altered to delete steps and/or include additional steps. The procedure begins by obtaining a test subject biological sample 302.

In certain embodiments, the biological sample can be, for example, whole blood, serum, plasma, tissue, saliva, a buccal swab, or urine. In certain embodiments, the biological sample can be dried and blotted onto a solid or porous substrate. In certain embodiments, an aliquot from a bulk sample is used to generate the vibrational spectrum. In certain embodiments, the biological sample is processed by one or more techniques such as, for example, filtration, centrifugation, ultracentrifugation, distillation, or chromatography.

In some embodiments, the test subject is suffering from chronic pain. In some embodiments, the test subject is a human. In some embodiments, the test subject is a mammal, such as a non-human mammal In some embodiments, the test subject is a human suffering from chronic pain who has visited a physician at least one, two, three, four, or five times in the past and has not received a diagnosis of the condition responsible for the chronic pain, or has not received a satisfactory diagnosis of the condition responsible for the chronic pain. A diagnosis of the condition responsible for the chronic pain is not satisfactory if attempts to treat the diagnosed condition have not noticeably decreased the chronic pain experienced by the test subject.

Next, the vibrational spectrum for the test subject biological sample is obtained (304). The spectrum can be generated by a Raman or FT-IR spectrometer, such as, for example, spectrometer 102. The spectrum can be acquired using using attenuated total reflection (ATR), transmission, or reflectance. The vibrational spectrum can be represented, for example, as an image, as raw data, as a list of peak magnitudes and locations (e.g., absorbance peaks, transmission peaks, and/or reflectance peaks).

Next, the vibrational spectrum can be prepared or processed (306). This processing can include one or more techniques such as, for example, transformation, normalization, truncation, clustering, classification, principal components analysis, linear regression, and nonlinear regression. This processing step can include any number of chemometric analyses such as, for example, Data Exploration and Pattern Recognition (Principal Components Analysis (PCA), Parallel Factor Analysis (PARAFAC), Multiway PCA); Classification (SIMCA, k-nearest neighbors, PLS Discriminant Analysis, Support Vector Machine Classification, Clustering (HCA)); Linear and Non-Linear Regression (PLS, Principal Components Regression (PCR), Multiple Linear Regression (MLR), Classical Least Squares (CLS), Support Vector Machine Regression, N-way PLS, Locally Weighted Regression); Self-modeling Curve Resolution, Pure Variable Methods (Multivariate Curve Resolution (MCR), Purity (compare to SIMPLSMA), CODA_DW, CompareLCMS); Curve fitting and Distribution fitting and analysis tools; Instrument Standardization (Piece-wise Direct, Windowed Piecewise, OSC, Generalized Least Squares Preprocessing); Advanced Graphical Data Set Editing and Visualization Tools; Advanced Customizable Order-Specific Preprocessing (Centering, Scaling, Smoothing, Derivatizing, Transformations, Baselining); Missing Data Support (SVD and NIPALS); and Variable Selection (Genetic algorithms, IPLS, Selectivity, VIP).

In certain embodiments, the vibrational spectrum may be additionally processed by, for example, subtracting the signal from water or a solvent, or isolating a particular region of the spectrum, such as the middle infrared spectrum. For example, the vibrational spectrum may be cropped to just 4,000-400 cm⁻¹, 4,000-600 cm⁻¹, or 2000-900 cm⁻¹.

Next, the vibrational spectrum or processed data is provided to classifier model (308). The classifier model may include, for example, an Artificial Neural Network, a decision tree, an expert system. In certain embodiments, a sequence of absorption peaks may be provided as an input feature vector to the Artificial Neural Network.

In certain embodiments, the classifier model may include one or more chemometric analyses. In certain embodiments, the classifier model may generate a classification that graphically or textually indicates the membership of the sample in one or more candidate chronic pain syndromes. In certain embodiments, the classifier model may calculate a fitness score or likelihood of the sample's membership in one or more candidate pain syndromes based on a measured similarity to the relevant training samples or spectral signature.

Next, the classification and/or one or more likelihoods (collectively, “results”) can be provided to the user (310). In certain embodiments, only a subset of the results are provided to the user. In certain embodiments, some or all of the results are displayed on a screen for a local user, or delivered to a remote user, or provided to a user by some other reasonable means. In certain embodiments, there may be one or more users.

Based on the results, a person, such as, for example, a medical professional, may provide instructions for evaluating the risk, predisposition, or prognosis of the one or more candidate chronic pain syndromes to a patient. In certain embodiments, the approach may additionally provide instructions for evaluating the risk, predisposition, or prognosis of the one or more candidate chronic pain syndromes. For example, the system may access a local or remote database collecting information about the risk, predisposition, or prognosis for any of the candidate chronic pain syndromes. The database may be modified based on particular patient outcomes and associate the observed risk, predisposition, or prognosis with a particular spectral signature. Such a database may additionally include information from the medical literature. In certain embodiments, a person, such as, for example, a medical professional, may obtain such instructions and provide the instructions for evaluating the risk, predisposition, or prognosis of the one or more candidate chronic pain syndromes to a patient. In certain embodiments, a person may access a database to obtain such instructions.

In certain embodiments, the approach may additionally provide suggested therapies for the one or more candidate chronic pain syndromes. For example, the system may access a local or remote database collecting information about therapies for any of the candidate chronic pain syndromes. The database may be modified based on particular patient outcomes and associate annotated successful or unsuccessful therapeutic approaches with a particular spectral signature. Such a database may additionally include information from the medical literature. In certain embodiments, a person, such as, for example, a medical professional, may administer a therapy to a patient based on the diagnosis and/or suggested therapies.

In certain embodiments, the approach may be used to generate statistical information for use in research, epidemiology, or insurance applications.

### EXAMPLE

The following specific example is to be construed as merely illustrative, and not limiting of the disclosure.

Twenty human serum samples, in liquid form, were analyzed by FT-IR using an Agilent Cary 670 spectrometer configured for the mid-infrared, with an extended range KBr beamsplitter and a high sensitivity linearized MCT detector. The samples consisted of 13 serum samples obtained from patients with fibromyalgia (“diseased”) along with 7 “healthy” serum samples. Samples were introduced to the FT-IR spectrometer via a diamond attenuated total reflectance (ATR) accessory providing “no sample preparation” acquisition of spectra. All data were collected and processed using RESOLUTIONS PRO FT-IR software (Agilent Technologies) with chemometric algorithms applied using PLS_TOOLBOX (Eigenvector Research, Inc.).

All sample spectra were collected using RESOLUTIONS PRO FT-IR software with no sample post-processing applied. Post-processing, clutter removal, and chemometric operations were performed using PLS_TOOLBOX. A water spectrum was subtracted from each sample spectrum.

Initial analysis was performed using Principal Component Analysis using four principle components in the model. The first and second principal components (PC1 and PC2) yielded substantially separable “diseased” and “healthy” groups. Specifically, it was possible to divide samples according to PC1 and PC2 such that all of the healthy samples were separated from twelve of the thirteen diseased samples, thus demonstrating approximately 92% sensitivity and perfect specificity for the classification of a sample as diseased in this small training set. Four of the seven healthy samples could be separated from all of the diseased samples.

The embodiments within the specification provide an illustration of embodiments of the invention and should not be construed to limit the scope of the invention. The skilled artisan readily recognizes that many other embodiments are encompassed by the invention.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the following claims.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described.

Other embodiments of the invention will be apparent to those skilled in the art from consideration of the specification and practice of the invention disclosed herein. It is intended that the specification and examples be considered as exemplary only.

1. A method for diagnosing a chronic pain syndrome in a test subject, the method comprising:
a. obtaining a vibrational spectrum for a biological sample from the test subject; and
b. classifying the spectrum as representative of one or more candidate chronic pain syndromes.

2. The method of claim 1, wherein the classifying step comprises:
a. calculating one or more scores assessing whether the test subject should be diagnosed with one or more candidate chronic pain syndromes from the output of a model, wherein the inputs to the model comprise the vibrational spectrum; and
b. providing the one or more scores.

3. The method of claim 1, wherein the biological sample is processed by a technique selected from the group consisting of: filtration, centrifugation, ultracentrifugation, distillation, or chromatography.

4. The method of claim 1, wherein the biological sample is whole blood, serum, plasma, tissue, or urine.

5. The method of claim 4, wherein the biological sample is dried and blotted onto a solid or porous substrate.

6.

7. The method of claim 1, wherein the one or more candidate chronic pain syndromes is selected from the group consisting of fibromyalgia, myofascial pain syndrome, complex regional pain syndrome, osteoarthritis, rheumatoid arthritis, and chronic fatigue syndrome, and Post-Traumatic Stress Disorder.

8. The method of claim 1, wherein the vibrational spectrum of the sample is obtained by utilizing a technique selected from the group consisting of: Fourier transform infrared spectroscopy, Raman spectroscopy or attenuated total reflection, transmission, or reflectance.

9. The method of claim 8, wherein the vibrational spectrum is processed by one or more techniques selected from transformation, normalization, truncation, clustering, classification, principal components analysis, linear regression, and nonlinear regression.

10. The method of claim 2, wherein the model comprises an artificial neural network.

11. The method of claim 2, wherein the model comprises processing by one or more techniques selected from clustering, classification, principal components analysis, linear regression, and nonlinear regression.

12. The method of claim 2, further comprising providing suggested therapies for the one or more candidate chronic pain syndromes.

13. A system for diagnosing a chronic pain syndrome in a test subject, the system comprising:
a. at least one device configured to generate a vibrational spectrum based on a biological sample;
b. one or more processors configured to execute one or more modules; and
c. a memory storing the one or more modules, the modules comprising instructions for:
i. obtaining the vibrational spectrum based on a biological sample;
ii. calculating one or more scores assessing whether the test subject should be diagnosed with one or more candidate chronic pain syndromes from the output of a model, wherein the inputs to the model comprise the vibrational spectrum; and
iii. providing the one or more scores.

14. The system of claim 13, wherein the at least one device is connected to the one or more processors via a network connection or a local data connection.

15. The system of claim 14, wherein the one or more candidate chronic pain syndromes is selected from the group consisting of fibromyalgia, myofascial pain syndrome, complex regional pain syndrome, osteoarthritis, rheumatoid arthritis, and chronic fatigue syndrome, and Post-Traumatic Stress Disorder.

16. The system of claim 15, wherein the vibrational spectrum of the sample is obtained by utilizing a technique selected from the group consisting of: Fourier transform infrared spectroscopy, Raman spectroscopy or attenuated total reflection, transmission, or reflectance.

17. A system for diagnosing a chronic pain syndromes in a test subject, the system comprising:
a. one or more processors configured to execute one or more modules; and
b. a memory storing the one or more modules, the modules comprising instructions for:
i. obtaining a vibrational spectrum based on a biological sample;
ii. calculating one or more scores assessing whether the test subject should be diagnosed with one or more candidate chronic pain syndromes from the output of a model, wherein the inputs to the model comprise the vibrational spectrum; and
iii. providing the one or more scores.

18. The system of claim 17, wherein the system is linked to a spectrometer.

19. The system of claim 14, wherein the one or more candidate chronic pain syndromes is selected from the group consisting of fibromyalgia, myofascial pain syndrome, complex regional pain syndrome, osteoarthritis, rheumatoid arthritis, and chronic fatigue syndrome, and Post-Traumatic Stress Disorder.

20. The system of claim 15, wherein the vibrational spectrum of the sample is obtained by utilizing a technique selected from the group consisting of: Fourier transform infrared spectroscopy, Raman spectroscopy or attenuated total reflection, transmission, or reflectance.
